(19) United States
(12) Patent Application Publication
(10) Pub. No.: US 2005/0027204 A1
(43) Pub. Date: Feb. 3, 2005

(54) ECG diagnostic system and method

(76) Inventors: Paul D. Kligfield, New York, NY (US); Robert Arzbaecher, Chicago, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 10/878,647

(22) Filed: Jun. 28, 2004

Related U.S. Application Data
(60) Provisional application No. 60/482,685, filed on Jun. 26, 2003.
(60) Provisional application No. 60/484,733, filed on Jul. 2, 2003.

(51) Int. Cl. A61N 1/37; A61B 5/0402

(52) U.S. Cl. 600/510; 607/27

(57) ABSTRACT

A system and method are disclosed for providing enhanced ECG diagnosis for patients having an implanted heart stimulation device. The system acquires data relating to the device for use in the ECG diagnosis. In one preferred form, data is transferred from an implanted transponder programmed with the data to a standard ECG monitoring electrode applied to the patient's chest, and the read out generated by the ECG machine includes the transferred data.

### FIELD OF THE INVENTION

This invention relates to a system and method of ECG diagnosis and more particularly, to an ECG diagnostic system and method for a patient with an implanted heart stimulation device.

### BACKGROUND OF THE INVENTION

Electrocardiographic (ECG) diagnosis is often difficult in patients with pacemakers, and it is becoming more difficult with increasing complexity of modern pacemaker function and with lead systems that reduce the magnitude of the pacemaker signal on the body surface. Diagnostic difficulties include simple detection of pacemaker presence, recognition of prevalent pacemaker mode, interpretation of pacemaker sensing and capture, and understanding of specialized pacemaker self-testing behavior. Preliminary computer-based misinterpretation of pacemaker function may mislead the electrocardiographer, whose ability to recognize normal and abnormal pacemaker behavior varies widely.

There is no present method by which pacemakers communicate their presence, basic identifying characteristics, and intrinsic mode settings to the standard 12-lead surface electrocardiogram. This limits the ability of the electrocardiographer to properly evaluate the status of pacemaker rate, sensing, and capture behavior.

Pacemakers function with a wide range of pacing, sensing, capture, chamber, and self-testing characteristics. Bipolar pacing leads and more efficient pacing power output reduce the magnitude of the pacemaker signal that is available for electrocardiographic recording on the surface of the body. Failure to detect or recognize pacemaker spikes on the surface ECG leads to errors of interpretation by the electrocardiographer. Failure to detect and to correctly interpret pacemaker signals by computer-based algorithms for preliminary interpretation can lead to a number of misleading suggestions to the electrocardiographer; in other cases, failure to detect and to correctly interpret pacemaker signals can decrease the ability of the electrocardiographer to detect pacemaker malfunction. Examples include, but are not limited to, the following:
When the ventricle is entirely paced in VVI or VVO mode, failure to detect and to correctly interpret the pacemaker activity can lead to a computer diagnosis of wide complex rhythm and intraventricular block (occasionally left bundle branch block), which mislead the electrocardiographer.
When the ventricle is entirely paced with fusion complexes in VVI or VVO mode, failure to detect and to correctly interpret the pacemaker activity can lead to false computer diagnosis of myocardial infarction and hypertrophy patterns, which mislead the electrocardiographer.
When the ventricle is partially paced in VVI mode, failure to detect and to correctly interpret the pacemaker activity can lead to computer misinterpretation of the underlying rhythm, misdiagnosis of ventricular ectopy, and inability to recognize pacemaker sensing and capture failure, each of which mislead the electrocardiographer.
When the ventricle is entirely paced in DDD mode in sinus rhythm, with and without ventricular fusion, failure to detect and to correctly interpret the pacemaker activity leads to a diagnosis of sinus rhythm, but with a variety of intraventricular block diagnoses, including axis shifts, which mislead the electrocardiographer.
When the heart is entirely paced in DDD (AV sequential) mode, failure to detect and to correctly interpret the atrial pacemaker activity leads to a diagnosis of sinus rhythm, while failure to interpret the ventricular pacemaker activity leads to a variety of intraventricular block diagnoses, including axis shifts, each of which mislead the electrocardiographer.
When the ventricle is partially paced in DDD mode, failure to detect and to correctly interpret the pacemaker activity can lead to computer misinterpretation of the underlying rhythm, misdiagnosis of ectopy, and inability to recognize pacemaker sensing and capture failure, each of which mislead the electrocardiographer.

Inability of the ECG to recognize features that are intrinsic to individual pacemaker function has additional clinical consequences. These include inability to properly interpret self-testing pacing threshold behavior that simulates failure to capture, which can mislead the electrocardiographer. Inability to detect pacemaker mode change can simulate inappropriate sensing behavior, which can mislead the electrocardiographer. Further, inability of the ECG to properly interpret the varied rate and mode changes that are associated with end of life behavior of multiple types of pacemakers limits the ability of the routine ECG to detect the need for pacemaker generator replacement.

Diagnostic skills with respect to recognition, understanding, and proper diagnosis of pacemaker behavior vary widely among primary interpreters of routine 12-lead electrocardiograms. There is no uniformly accepted or applied credentialing standards for physician interpreters of electrocardiograms in the United States. The limitations and problems outlined above can mislead experienced electrophysiologists, and they are increasingly problematic for general cardiologists, general internists, and emergency room physicians who are responsible for routine and emergency interpretation of electrocardiograms.

The importance of the problem is indicated by the large number of electrocardiograms that contain pacemaker signals. It has been estimated that over 100,000,000 routine 12-lead ECGs are performed and interpreted annually in the United States. Worldwide application is approximately three times that number. Pacemaker activity, which will be interpreted either correctly or incorrectly by the electrocardiographer, is present in an estimated 1-5% of these routine tracings.

It can be estimated that 200,000 cardiac pacemakers, separate from and in addition to newer implantable cardiac pacer-defibrillators, are implanted annually in the United States for control of bradyarrhythmias or atrio-ventricular heart block. Based on an average of 8 years of pacing per patient, the total adult population with implanted pacemakers in the United States approaches 1,600,000. This represents approximately 1% of the adult population of the United States who are routine candidates for standard electrocardiography. Pacemaker implantation in Europe is performed at approximately one-half the rate as in the United States, while rates in other parts of the world are generally lower and vary widely.

Since patients with implanted pacemakers are already identified as subjects with increased cardiovascular disorders, routine electrocardiography would be expected to be at least twice as common in this group as in the total general adult population of the United States, which includes healthy subjects. In overall clinical practice, therefore, the proportion of routine 12-lead electrocardiograms performed in patients with pacemakers can be estimated to be 2%, or 2,000,000 routine tracings, each year.
In hospital-based practice, where the prevalence of patients with cardiac disease exceeds that of the general adult population, the proportion of routine 12-lead electrocardiograms performed in patients with pacemakers might be increased several-fold, perhaps approaching 6% of hospital-based tracings.
Evidence of pacemaker implantation on routine electrocardiograms will not be present in patients with demand mode pacing in whom pacing is normally inhibited. Even so, the presence of underlying pacemaker sensing and function in these patients would be useful clinical information that is currently not available during electrocardiography. Allowing for variable normal pacemaker inhibition, it can be estimated that pacemaker activity might be present in 1-5% of routine electrocardiograms interpreted in the United States, with the prevalence varying with concentration of patients at risk in different clinical settings.
This estimate is supported by analysis of electrocardiographic diagnoses for a recent 6 year period at a tertiary care teaching hospital in the northeastern United States. Pacemaker activity was recorded and detected in 4.3% of routine tracings, representing 16,043 instances among 376,511 electrocardiograms.

In addition to traditional single- and dual-chamber implantable pacemakers that are used predominantly for control of bradyarrhthmias, pacemaker functions are intrinsic components of newer implantable devices for the control of cardiac rhythm. Recognition of these devices is essential for proper interpretation of their effect, when present, on the routine electrocardiogram. The increasing use of these devices will increase the proportion of routine 12-lead electrocardiograms that contain pacemaker signals.

Both single- and dual-chamber pacing modalities are now combined with anti-tachycardia pacing and defibrillating modalities of newer implantable cardioverter-defibrillators. Based on evolving studies, it has been estimated that 300,000-600,000 of these devices might be implanted each year in the United States alone. This would add significantly to the number of electrocardiograms in which device data would improve interpretation by the electrocardiogapher.

Other newer devices can be anticipated to have prevalent effects on interpretation of the 12-lead electrocardiogram because of the large number of patients for which they are being targeted. Cardiac re-synchronization therapy via bi-ventricular pacing is increasingly used for the important and highly prevalent problem of congestive heart failure. Recognition of the intended performance of these devices would improve interpretation by the electrocardiographer. Implantable devices for management of atrial arrhythmias also require recognition and proper interpretation.

Commercially available pacemakers have the ability to communicate with an external programmer. A “programming head” connected to the external programmer is placed on the skin of the patient, directly over the implanted pacemaker, and electromagnetic communication is established via antennas in both the programming head and the pacemaker. At the time of implant or during a follow-up session the physician can, from the keyboard of the programmer, determine the battery status and interrogate or change the mode of pacing and the pacing parameters. In addition, the pacemaker can transmit to the programmer, in real time, signals consisting of the intracardiac electrograms being sensed by the implanted pacing leads, as well as marker pulses delineating the time at which events are detected or pacing stimuli are delivered by the pacemaker. The programmer has the ability to display or print the above-described data and signals.

The aforementioned programmers are complex and costly, because they have a dual function: transmitting programming data to the implanted pacemaker and receiving and recording programmed data and signals from the implanted pacemaker. To perform these functions, each programmer will contain two-way data transfer or communication capability, keyboard, visual display, printer, and chart recorder. Additionally, the aforementioned programmers are designed specifically to work with a particular manufacturer and type of pacemaker, and cannot be used with pacemakers of another manufacture or model.

It is clear from the above that the aforementioned programmers are suitable for use in pacemaker implantation and follow-up reprogramming by physicians specializing in the care of pacemaker patients, but are too complex and costly to be available and useful to medical staff who are simply recording or interpreting a routine electrocardiogram. Herein, a need has been identified for a simpler device that can be available, such as whenever an electrocardiogram is ordered, to determine, first, if the patient has a pacemaker and, second, to interrogate the pacemaker and send pacemaker data to the electrocardiograph for display and recording.

### SUMMARY OF THE INVENTION

In accordance with one aspect of the invention, a system and method are provided that enable communication of data or information related to a heart stimulation device, e.g., an implanted cardiac pacemaker or defibrillator, to an external reader as may be incorporated in an electrocardiograph apparatus to improve interpretation capability of the electrocardiographer and to enhance preliminary computer-based analysis of pacemaker function by the electrocardiograph. Transmitted information may include, but is not limited to, pacemaker identification, active mode of behavior, and sensing and pacing markers.

In a preferred form, the data or information communicated to electrocardiographs include the heart stimulation device manufacturer, type, pacing mode, programmed pacing parameters, and sensing/pacing event markers.

As earlier discussed, erroneous diagnosis of pacemaker sensing and capture adversely affects patient safety and care. When recognized with the system and method herein, pacemaker undersensing or oversensing can be corrected to regulate cardiac rhythm in the prescribed manner. Appropriate recognition of failure to pace or to capture can prevent life-threatening bradyarrhythmias in susceptible patients. Detection and recognition of occult pacing or pacing fusion can eliminate erroneous diagnoses of conduction block, axis deviation, and myocardial infarction. Detection and recognition of normal intermittent pacemaker function, previously undetectable on the surface electrocardiogram, can improve interpretation of repolarization changes associated with pacemaker memory effect. Anticipation of pacemaker battery end-of-life can prompt needed generator replacement that might be prevented by failure to undergo regular transtelephonic or office based pacemaker monitoring. The system and method herein employed, in one aspect, routine 12-lead electrocardiography as an efficient location for communication of pacemaker behavior. Accurate recognition of pacemaker function and dysfunction during routine electrocardiography will reduce the risk of serious arrhythmic events, which in turn should be cost-effective, in patients with heart disease.

### BRIEF DESCRIPTION OF THE DRAWINGS

FIG. 1 is a flowchart showing a method of acquiring data from an implanted heart stimulation device for use in diagnosing an ECG in accordance with the invention;

FIG. 2 is a perspective view of one form of a system for performing the method of FIG. 1 showing an ECG apparatus that receives the heart stimulation device data;

FIG. 3 is an ECG showing a printout from the ECG apparatus including the heart stimulation device data, ECG traces, and a marker channel denoting the time of sensed and paced events in the heart; and

FIG. 4 is a diagram of the system of FIG. 2 and showing components of a heart stimulation device in the form of a pacemaker.

### DETAILED DESCRIPTION OF THE PREFERRED EMBODIMENTS

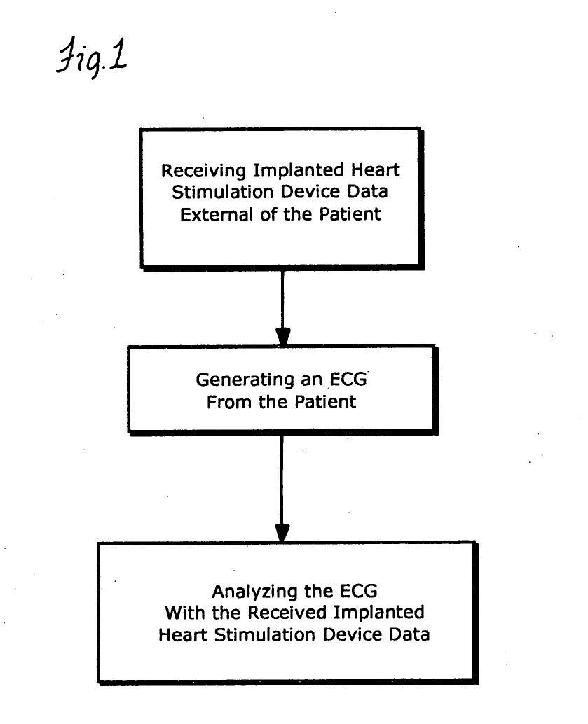

The simplest system embodiment of implementing the method of FIG. 1 employs an implanted transponder and external handheld reader, such as are well known in the radio frequency identification (RFID) art for tagging free-ranging animals, for example. The implanted transponder contains an integrated circuit device or electronic chip or chip set programmed with data or an identifying code and a tightly wrapped coil antenna, which are enclosed in an hermetic capsule less than one-tenth inch in diameter and one-half inch in length. The external reader produces a low frequency magnetic field via its antenna, and the transponder is excited by the magnetic field, causing it to transmit its identifying code via information signals back to the reader. Circuitry for interpreting these signals can be provided in various components used to acquire the heart stimulation device data, such as in the reader or, alternatively, further downstream therefrom such as in the ECG machine 12, or in both. Herein, the terms data or information can also broadly mean signals coded with the data or information so that they are data or information carrying signals. The transponder functions can be integrated into the pacemaker or alternately, the transponder can be implanted separately, using a device similar to a hypodermic syringe to inject it under the patient's skin. It requires no battery since it is powered by the magnetic field emitted by the reader. The distance at which the reader can excite and interrogate the transponder is of the order of 12-20 inches; thus the person recording the ECG need not know precisely where in the upper thorax a pacemaker may have been implanted.

The transponder can be pre-programmed with up to 200 bits of information describing the pacemaker: manufacturer, type, possible modes of pacing. When the reader is brought into proximity with the transponder it automatically displays on its screen a listing of the programmed information. The existence of a pacemaker as well as the programmed data can then be entered into the electrocardiographic record, either automatically by downloading from the reader, or manually by the technician. In the automatic version, the reader apparatus is preferably incorporated with the ECG apparatus that generates the ECG. These are often the only data needed by the electrocardiographer to correctly interpret the paced ECG.

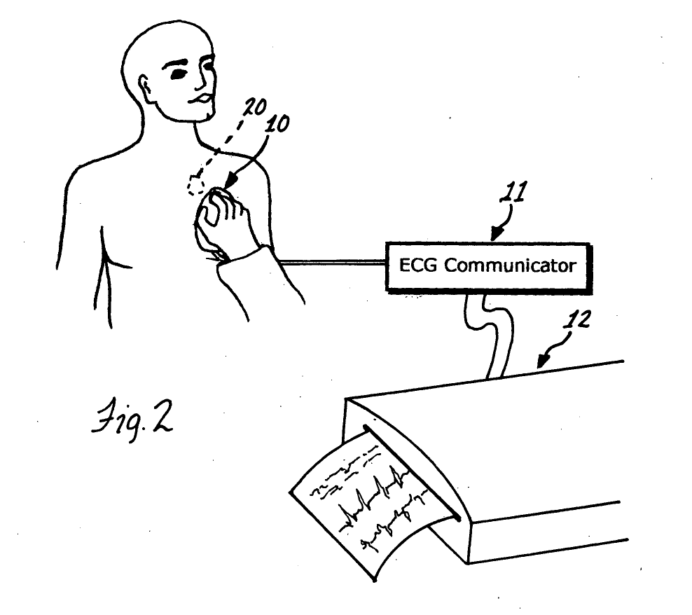

A second embodiment, one which permits a more detailed interrogation of an implanted pacemaker, can be described with reference to FIG. 2. A patient is shown in whom a pacemaker 20 has been implanted. Also shown is an electromagnetic wand 10 that is designed such that it can be placed on the skin directly over the implanted pacemaker. The wand and pacemaker each have coils of wire contained within them that serve as antennas in close proximity to each other when the wand is properly placed. In operation, the button on the wand is depressed to command a transmission by the pacemaker. The pacemaker receives the command and transmits data back to the wand. These data may include, but are not limited to: pacemaker manufacturer and type; pacing mode, whether DDDR, DDD, DDIR, DDI, VVIR, VVI, AAIR, AAI, or other; any special features that have been programmed on, such as antitachycardia, rate responsive, autocapture, mode switch, PVC response, etc; pacing parameters including lower pacing rate, upper tracking rate, upper sensor rate, atrial and ventricular pace blanking, pace AV interval, sense AV interval, post-ventricular atrial refractory period, atrial refractory period, etc. Following the transmission of this status information, the pacemaker transmits marker signals synchronous with the sensing of atrial and ventricular events and the delivery of atrial and ventricular pacing stimuli.

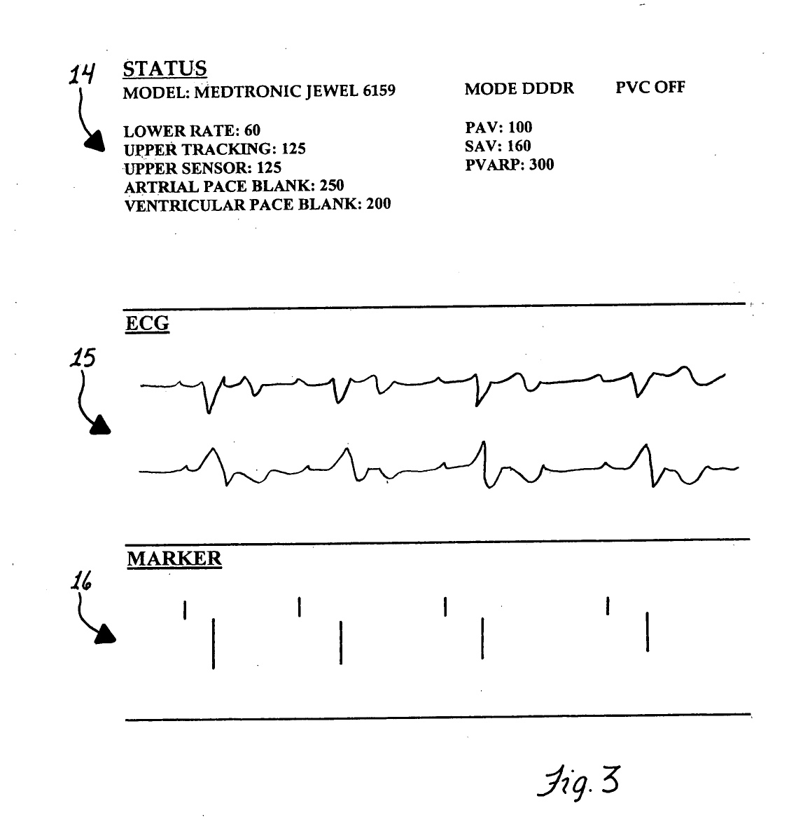

The above-described transmission from the pacemaker is received by the wand and delivered to an external reader apparatus in the form of an ECG communicator 11 that is designed to interpret the signal and forward it to an electrocardiograph machine 12 for display and printing. An example of a printed page is shown in FIG. 3. The page contains pacemaker status information 14, electrocardiogram traces 15, and a marker channel 16 denoting the time of sensed and paced events in the atria and ventricles. Other formats are also anticipated, including a continuous recording of several pages for displaying more than a few seconds of electrocardiogram and marker data. The communicator 11 transfers pacemaker data or information to the machine 12 via link 30, that is either internal to the machine 12 or ECG apparatus so that the communicator 11 is integrated therewith, or external of the machine 12 so that the communicator 11 is a separate module and together with the ECG machine 12 forms an ECG apparatus.

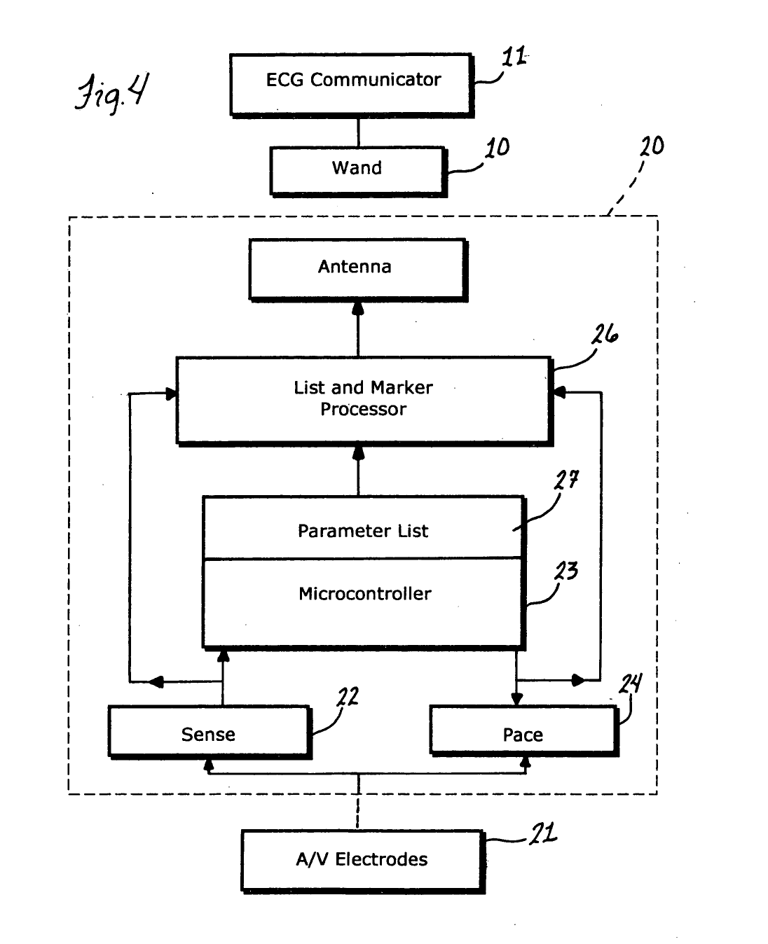

Further elaboration of the invention can be understood by referring to the pacemaker components in FIG. 4, which would be familiar to someone skilled in the art. Intracardiac electrodes 21 in the atrium and ventricles of the heart are connected to the input sense amplifiers 22 wherein the signals are amplified and digitized. The digital data are then processed by microcontroller 23, which analyzes the timing of all atrial and ventricular sensed events, delivers pacing stimuli when such timing does not violate the restrictions specified by the aforesaid programmable parameters, and withholds the deliver of pacing stimuli when the timing violates those restrictions. The parameters that are consulted by the microcontroller are stored in a section of microcontroller memory known as a parameter list 27. When the aforementioned interrogate command is received from the wand the contents of the parameter list are acquired by the list and marker processor 26 and transmitted to the wand and thence to the ECG communicator for printing by the electrocardiograph machine. After this transmission the list and marker processor transmits, in real time, the signal marking the occurrence of each sensed and paced event, and this signal is also recorded by the electrocardiograph machine.

In a third embodiment, the wand is eliminated and the pacemaker transmits status data and marker signal directly to the ECG communicator by radio frequency (RF) transmission using the pacemaker power source. The distance between the patient and said ECG communicator in this embodiment could be, for example, 10 feet. The transmitter frequency could be, for example, 420 MHz, a frequency designated by FCC for medical applications. A command to transmit said data and signal is transmitted directly from the ECG communicator to the implanted pacemaker, using the same frequency.

In a fourth embodiment, the pacemaker status and marker data are not transmitted, but are superimposed on a suitable carrier and delivered directly to the pacemaker intracardiac electrodes. The signal power is well beneath that of a pacing stimulus and has no physiological effect. However, the signal can be detected using electrodes on the skin, including in particular the standard ECG electrodes. In this embodiment, the cable from the standard ECG electrodes is connected directly to the ECG communicator, which has additional receiver circuitry for separating the high-frequency carrier-based status and marker signal from the lower-frequency electrocardiographic signal. The status data, marker signal, and ECG are then delivered to the ECG machine for printing.

As is apparent, the systems disclosed herein all include information or data acquiring means that allow an electrocardiographer to easily acquire information or data relating to the implanted heart stimulator device, e.g., pacemaker or defibrillator, to enable correct analysis or diagnosis of a paced ECG, whose pacing may otherwise be undectable or simply undetected by the electrocardiographer. The systems only vary in how the data is acquired and, to this end, the amount of additional hardware needed for this purpose over that already present for the taking of an ECG from a patient. Accordingly, in some embodiments, the information or data is acquired from a separate transponder, and in others the data is received from the implanted, heart stimulation device to keep system hardware requirements to a minimum. In addition several ways to acquire the information signals are also described, either by a reader or wand apparatus that can detect the signals emitted by the transponder or heart stimulation device, by the ECG communicator by having the signals emitted via an RF frequency, or by using the ECG apparatus electrodes applied to the patient's chest.

In another version of an ECG diagnostic system in accordance with the method of FIG. 1 as employed by the previously described systems, the reader apparatus is incorporated with ECG apparatus by including its antenna with the ECG electrodes. More specifically, the reader apparatus is integrated into the ECG machine, and its antenna is on or within one or more of the ECG electrodes that are routinely attached to the chest during an ECG procedure. Accordingly, one or more of these chest electrodes will be close enough to the implanted transponder to energize it and receive its data identifying the implanted heart stimulator device and its operating characteristics. Thus, the person recording the ECG need not know where in the upper thorax the transponder may have been implanted. When the ECG electrodes are applied to the chest in the usual manner and the ECG machine is turned on, the reader circuitry in the ECG machine is operable to automatically energize the implanted transponder, read and interpret the information signals emitted therefrom and deliver this information to the ECG machine for display and/or printing.

While the foregoing described embodiments have been set forth above, it will be appreciated to one skilled in the art that the inventions described have applications beyond the described embodiments. Accordingly, it is intended that the scope of the invention including such alternatives, modifications, and variations contemplated shall be defined by the appended claims.

1. A system for providing information relating to a heart stimulation device implanted in a body of a patient, the system comprising:
a reader apparatus external of the patient's body;
information signals carrying data relating to the implanted heart stimulation device distinct from stimulation signals generated by the heart stimulation device for stimulating the heart; and
circuitry of the reader apparatus configured to interpret the information signals for providing heart stimulation device data for use in analysis of an electrocardiogram (ECG).

2. The system of claim 1 wherein the reader apparatus comprises an ECG apparatus.

3. The system of claim 2 wherein the ECG apparatus includes an ECG electrode that receives the information signals.

4. The system of claim 2 wherein the ECG apparatus includes an ECG machine for generating an ECG and an ECG communicator connected to the ECG machine and which is adapted for wireless communication, and the information signals are RF signals for being received by the ECG communicator.

5. The system of claim 1 including an ECG machine for generating the ECG and an ECG link between the reader apparatus and the ECG machine for transferring the heart stimulation device data to the ECG machine.

6. The system of claim 5 wherein the ECG link is internal to the ECG machine so that the reader apparatus is integrated therewith.

7. The system of claim 1 wherein the reader apparatus comprises a hand-held transmitter for initiating transfer of the data thereto, and a screen for displaying the transferred data.

8. The system of claim 1 in combination with the heart stimulation device wherein the heart stimulation device generates both the information and stimulation signals.

9. The combination of claim 8 wherein the heart stimulation device includes at least one intracardiac electrode that carries the information and stimulation signals with the information signals having a predetermined characteristic so that the information signals do not affect heart functions, and
an ECG machine that includes at least one external ECG monitoring electrode for detecting both the information and stimulation signals and transmitting the signals to the ECG machine.

10. The system of claim 1 including an implantable transmitter having circuitry configured to be programmed with the data and for transmitting the information signals to the reader apparatus.

11. The system of claim 10 in combination with the heart stimulation device wherein the transmitter is integrated with the heart stimulation device.

12. The system of claim 10 wherein the transmitter is a compact transponder implanted in the patient's body and is separate from the heart stimulation device.

13. The system of claim 10 wherein the external reader apparatus includes a power source that powers both the reader apparatus and the transmitter.

14. The system of claim 1 wherein the data relating to the heart stimulation device comprises manufacturer, type of heart stimulation device and pacing mode information of the device.

15. The system of claim 14 wherein the data further includes programmable features of the heart stimulation device.

16. The system of claim 1 in combination with the heart stimulation device wherein the device comprises a pacemaker, and the stimulation signals comprise electrical pacing pulses.

17. An ECG and pacemaker system comprising:
a pacemaker for pacing a heart;
an ECG machine for generating an ECG relating to the paced heart; and
means for acquiring data relating to the pacemaker for ECG diagnosis.

18. The system of claim 17 wherein the data acquiring means comprises an implanted device programmed with the data and at least one ECG electrode of the ECG machine that receives the programmed data from the implanted device.

19. The system of claim 18 wherein the implanted device comprises a compact, implanted transponder.

20. The system of claim 18 wherein the implanted device comprises the pacemaker.

21. The system of claim 17 wherein the data acquiring means comprises information signals generated by the pacemaker and an ECG communicator that interprets the information signals from the pacemaker and transfers the data to the ECG machine.

22. The system of claim 21 wherein the data acquiring means further comprises either a wand or at least one ECG electrode of the ECG machine for receiving the pacemaker information signals.

23. An ECG diagnostic system for analyzing paced ECGs of patients including implanted heart stimulation devices, the system comprising:
a compact, implanted device separate from a heart stimulation device;
programmable circuitry of the device for being programmed with data relating to the separate, implanted heart stimulation device;
an ECG electrode adapted for being attached to a patient having the implanted devices and receiving the heart stimulation device data; and
an ECG apparatus including the ECG electrode that displays an ECG and the heart stimulation device data.

24. The system of claim 23 wherein the compact, implanted device is a transponder having an integrated circuit device including the programmable circuitry and an antenna, and the ECG electrode includes an antenna.

25. The system of claim 23 wherein the ECG apparatus includes an ECG communicator that includes circuitry adapted for receiving the heart stimulation device data and initiating transfer of the data from the implanted device.

26. The system of claim 25 wherein the ECG apparatus includes an ECG machine having the ECG communicator integrated therewith.

27. The system of claim 23 wherein the compact, implanted device has a diameter of less than approximately one-tenth of an inch and a length of approximately one-half inch.

28. An ECG diagnosis method for use with patients having an implanted heart stimulation device, the method comprising:
receiving data relating to the implanted heart stimulation device with an apparatus external of the patient's body;
generating an ECG from the patient; and
analyzing the ECG with the received data allowing enhanced accuracy in the ECG analysis.

29. The method of claim 28 including programming a transponder with the data, and implanting the transponder in the patient.

30. The method of claim 28 including transferring the received data to an ECG machine that generates the ECG.

31. The method of claim 30 including displaying an ECG trace and the received data relating to the heart stimulation device.

32. The method of claim 30 wherein the received data comprises coded signals,
interpreting the received coded signals with an intermediate communicator, and transferring the data to the ECG machine.

33. The method of claim 30 wherein the apparatus external of the patient's body is an ECG machine, and data is received by sensing signals coded with the data with at least one ECG electrode.

34. The method of claim 28 wherein the device data are received and the ECG is generated at substantially the same time.
